(19) United States
(12) Patent Application Publication
(10) Pub. No.: US 2011/0196358 A1
(43) Pub. Date: Aug. 11, 2011

(54) CLOSED ULTRA-RAPID CELL VITRIFICATION DEVICE AND SEALING PROCEDURE OF THE DEVICE

(76) Inventor: ENRIQUE CRIADO SCHOLZ, Lopera (ES)

(21) Appl. No.: 13/022,486

(22) Filed: Feb. 7, 2011

(30) Foreign Application Priority Data
Feb. 9, 2010 (ES) P 201030167

(51) Int. Cl. A61B 18/02

(52) U.S. Cl. 606/20

(57) ABSTRACT

The present invention provides a closed ultra-fast device for vitrification that reduces the risk of contamination; favors and increases the survival of human cells (e.g., oocytes, embryos, sperm, etc.) or non-human cells after thawing; and achieves ultra-fast cooling rates with a low concentration of cryo-protectors. The device of the present invention avoids risk of contamination, favors and increases the survival rate of human (oocytes, embryos or sperm, etc) or nonhuman cells after thawing, featuring ultra-rapid cooling rates and the use of low concentrations of cryo-protectors. The device comprises a protective sheath made of an inert, flexible and transparent material, inside of which a micro-capillary, preferably of quartz, is intended to house the cells that are to be vitrified. The protective sheath is adapted to be protectively sealed at its superior extremity, thereby creating a hermetic seal of the device and preventing the entry of coolant (liquid nitrogen, slush or slurry) into the protective sheath.

### CROSS REFERENCE TO RELATED APPLICATION

This application claims the benefit of Spanish Patent Application P 201030167, filed Feb. 9, 2010, which is hereby incorporated by reference herein in its entirety.

### FIELD OF THE INVENTION

The present invention relates to the field of culture or microorganism conservation and more specifically to the preservation of human, animal or plant cells.

### BACKGROUND OF THE INVENTION

There are two techniques to preserve cells: slow freezing and vitrification.

Slow freezing is based on controlling the cooling rate in order to create a balance between the various factors that cause cellular damage, among which are the formation of ice, fractures and excessive dehydration of the cell.

In 1986, C. Chen achieved the first ever birth obtained from cryopreserved human oocytes following the application of this method, using a freezing protocol based on the addition of dimethyl sulfoxide (DMSO). Since then the results have varied greatly, obtaining a low survival rate due to the intracellular formation of ice crystals or other damage such as, the alteration of the mitotic spindle and zona pellucida. The freezing protocol starts at room temperature reaching values up to −150° C. Each stage is performed with various decreasing rates in temperature, ranging from −2° C., in the first stage, to −50° C. in the final stage. Finally. the capillaries containing the cells are transferred into tanks of liquid nitrogen at −196° C. for storage.

Vitrification is a procedure whereby liquid is solidified in a vitreous phase (not crystalline) with a rapid decrease in temperature and an increase in viscosity, avoiding the toxicity and formation of intracellular ice crystals that could damage the cell content. Several factors affect the probability of achieving an adequate vitrification, such as: cooling and heating rhythms, viscosity of the sample and volume of the sample.

Various methods exist to achieve vitrification, all using a high concentration of cryo-protecting agents (ethylene glycol, dimethyl sulfoxide, 1,2-propanediol, etc) reaching levels of 8M in some protocols. These cryo-protectors are very toxic to the cell at high concentrations and over long periods of exposure. Very high cooling rates are necessary, in the order of tens of thousands of degrees per minute, immersing the sample directly into liquid nitrogen.

One of the vitrification techniques developed in recent years uses an open system of micro capillaries, of 0.1 to 0.4 mm internal diameter and 0.01 mm thick, as well as liquid nitrogen “slush”, which is sub-cooled liquid nitrogen that presents a temperature of −210° C., lower than liquid nitrogen which is −196° C. to achieve an increase in cooling rates, achieving Speeds of up to 250,000° C. per minute, and consequently the possibility to reduce the concentration of cryo-protectors to 2M, only slightly toxic to the cell, increasing the possibility of development after thawing.

However, the former technique has the major inconvenience that contamination exists in open systems, the cells are in direct contact with liquid nitrogen. and stored in a single tank or container with other cryo-preserved cells.

Accordingly, it is desirable to develop an effective system for sealing the micro-capillary that can also withstand cryogenic temperatures.

### SUMMARY OF THE INVENTION

The present invention provides a closed ultra-fast device for vitrification that reduces the risk of contamination; favors and increases the survival of human cells (e.g., oocytes, embryos, sperm, etc.) or non-human cells alter thawing; and achieves ultra-fast cooling rates with a low concentration of cryo-protectors.

The present invention also provides a method for sealing a closed ultra-fast cell vitrification device.

### BRIEF DESCRIPTION OF THE FIGURES

Various objects, features and advantages of the present invention can be more fully appreciated with reference to the following detailed description when considered in connection with the following drawings, in which like reference numersal identificy like elements.

FIG. 1 shows an overview of the ultra-rapid closed cell vitrification device, according to an embodiment of the present invention.

FIG. 2 shows a perspective view of the micro-capillary according to an embodiment of the present invention.

FIG. 3 shows a sectioned view of a device according to an embodiment of the present invention. The device has been hermetically closed and prepared to be deposited in the general container of liquid nitrogen where all the other cells are stored.

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

The present invention provides a closed ultra-fast device for vitrification. The device reduces the risk of contamination, favors and increases the survival of human cells (e.g., oocytes, embryos, sperm, etc.) or non-human cells after thawing and achieves ultra-fast cooling rates with using low concentrations of cryo-protectors.

The closed cell vitrification device of the present invention comprises a protective sheath, closed at its inferior extremity, preferably made of an inert, flexible and transparent material. The interior is intended to house a quartz micro-capillary containing the cells that will be vitrified and the protective sheath is adapted to be sealed at its superior extremity, thus establishing a hermetic seal of the device and preventing the entry of coolant into the protective sheath when placed in a storage container.

The coolant can be liquid nitrogen, which has a temperature of −196° C., “slush” or sub-cooled liquid nitrogen which has a temperature of −210° C. or “slurry” which is a mixture of liquid nitrogen with different particles, such as copper powder or sodium chloride, depending on the characteristics of the cells to cryo-preserve.

Sealing the superior extremity of the protective sheath can be achieved by ultrasonic sealing, or by applying heat using a heat seal, or by a radio-frequenzy seal.

Preferably, the protective sheath consists additionally of a weight element placed on the inferior extremity, which prevents buoyancy once immersed in the liquid nitrogen. The protective sheath has the capacity to resist very low temperatures, as well as the great expansion pressures exerted by the coolant.

The protective sheath can have identification labels resistant to the coolant, or a sufficient area in which to write references or identification numbers.

All materials used to manufacture the protective sheath and micro-capillary are biocompatible and adapted to be sterilized by irradiation, thus guaranteeing and ensuring their use with human cells.

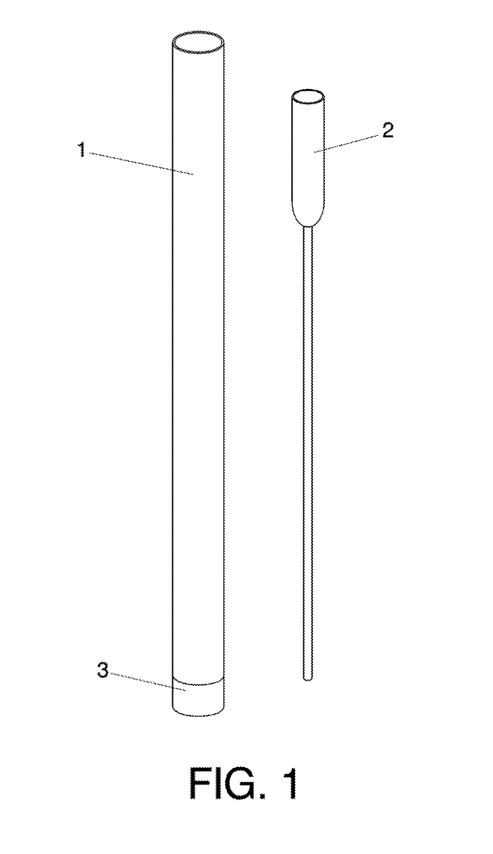

As shown in FIG. 1, the ultra-rapid closed cell vitrification device comprises a protective sheath (1) made of lonomeric resin, closed at its inferior extremity, and whose interior is designed to house a quartz micro-capillary (2) containing cells (4) that are to be vitrified. The protective sheath (1) is adapted to be sealed at its superior extremity, establishing a hermetic seal of the device and preventing entry of liquid nitrogen into the protective sheath (1).

Also, as seen in FIG. 1 and FIG. 3, the protective sheath (1) further comprises a weight element (3) located at the inferior extremity. The weight element (3) prevents buoyancy of this once introduced into the liquid nitrogen.

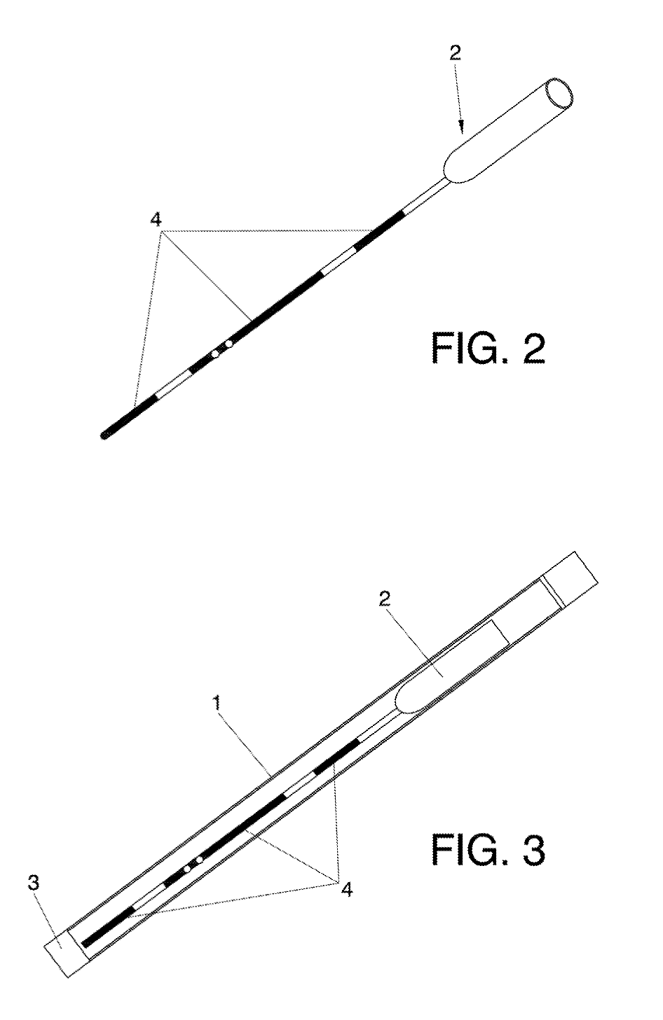

FIG. 2 shows a perspective view of the micro capillary (2), where the cells (4) contained within, are deposited leaving three air spaces between them, in order to prevent further contamination between cells (4).

FIG. 3 shows the device hermetically closed, with the micro-capillary (2) placed in the protective sheath (1), ready to be deposited in the general container of liquid nitrogen and kept there where the cells (4) will be stored along with many others without contact between them, thus avoiding contamination.

Using quartz micro-capillaries (2) to achieve high speeds of cooling and warming is motivated by the high thermal conductivity of quartz (6.5 W/mK, compared to the PVC of OPS capillaries, around 0.19 W/mK), as well as by the small inner diameter (between 0.1 mm and 0.4 mm) and the small size of the wall (0.01 mm) that can be achieved by the current state of techniques for this material. However, this micro-capillary (2) can be of any other material which has high thermal conductivity (e.g., plastic, glass, stainless steel, sapphire, gold, diamond, titanium, palladium, platinum, silver, etc).

The present invention also provides a procedure or method for sealing the closed vitrification device, once the cells are passed through the cryo-protective agents, and introduced into the micro-capillary. The method comprises four steps:
(a) immersing the protective sheath in a coolant solution (e.g., liquid nitrogen, slush or slurry). The immersing step is preferably performed completely, filling the protective sheath with coolant solution.
(b) introducing the micro-capillary into the protective sheath with the help of tweezers, allowing the micro-capillary to reach the bottom of the sheath.
(c) extracting the superior extremity of the protective sheath above the surface of the coolant solution, about 3 cms, to determine the heating of the superior extremity by the room temperature, and evaporating the coolant solution contained in the interior.
(d) Seal the superior extremity of the protective sheath, thereby ultrasonic sealing or by applying heat with a heat seal.

Once the device is hermetically closed, it can be then transferred and deposited into a general container. In the general container, the cells can be stored along with many others. without coming into contact, thus avoiding contamination.

What is claimed is:

1. A closed ultra-fast cell vitrification device comprising a protective sheath, sealed at the inferior extremity, the interior is intended to house a micro-capillary which will contain a number of cells for vitrification,
the protective sheath is adapted to be protectively sealed at its superior extremity, to establish a hermetic seal of the device and prevent entry of coolant into the protective sheath.

2. The closed ultra-fast cell vitrification device according to claim 1, wherein the protective sheath further comprises a weigh element at the inferior extremity, which avoids buoyancy once submerged in the coolant.

3. The closed ultra-fast cell vitrification device according to claim 1 or 2, wherein the protective sheath is made of an inert, flexible and transparent material.

4. The closed ultra-fast cell vitrification device according to claim 1 or 2, wherein the protective sheath (1) is made of ionomeric resin.

5. A method for sealine a closed ultra-fast cell vitrification device, comprising:
(a) immersing the protective sheath in a coolant solution, wherein the protective sheath is filled with coolant solution,
(b) introducing a micro-capillary into the protective sheath,
(c) extracting the superior extremity of the protective sheath above the surface of the coolant, approximately 3 cms, to determine the heating of the superior extremity of the protective sheath by the room temperature, and evaporation of the coolant contained in the interior, and
(d) sealing the superior extremity of the protective sheath,
wherein the closed ultra-fast cell vitrification device is as defined in any one of claims 1-4.

6. The method according to claim 5, wherein the protective sheath is completely immersed in the coolant solution.

7. The method according to claim 5 or 6, wherein the coolant solution is liquid nitrogen, slush or slurry.

8. The method according to claim 5 or 6, wherein the superior extremity of the protective sheath is extracted approximately 3 cms above the surface of the coolant solution.

9. The method according to claim 5 or 6, wherein the sealing is done ultrasonically.

10. The method according to claim 5 or 6, wherein the sealing is done by applying heat with a heat seal.

11. The method according to claim 5 or 6, wherein the sealing is done by a radio-frequency seal.
